(19) United States
(12) Patent Application Publication
(10) Pub. No.: US 2011/0201102 A1
(43) Pub. Date: Aug. 18, 2011

(54) Method and apparatus for cultivating organisms

(76) Inventor: Charles Wilson Harris, Phoenix, AZ (US)

(21) Appl. No.: 12/929,809

(22) Filed: Feb. 17, 2011

Related U.S. Application Data
(60) Provisional application No. 61/282,475, filed on Feb. 17, 2010.

(51) Int. Cl. C12M 1/02

(52) U.S. Cl. 435/292.1; 435/289.1

(57) ABSTRACT

A bioreactor for growing organisms such as microalgae, includes a container having a parabolic cross section for carrying a culture medium for the organism. A generally triangular flow director in the bottom center of the container with a tube at its top end contains openings for directing fluid such as a growth medium under pressure upwardly into the culture medium to create laminar flow of the culture medium in counter rotating paths of travel on opposite sides of the flow director. Thus, thorough mixing of the culture medium and organism is achieved.

The present invention provides a bioreactor system which ensures continuous, thorough mixing of organisms such as microalgae, growth medium and nutrients, and constant changing of the areas or zones of the biomass and movement to the available light, resulting in an enhanced growth environment for the organisms.

### FIELD OF THE INVENTION

This invention relates to a bioreactor system for growing organisms, and in particular microalgae.

### DESCRIPTION OF RELATED ART

Oil extracted from microalgae is used to produce biodiesel and pharmaceutical products. Systems for growing microalgae are disclosed, for example, in US Patent Applications 2008/0220515 (McCall), published Sep. 11, 2008; 2008/0268302 (McCall), published Oct. 30, 2008; 2008/0274494 (Kertz), published Nov. 6, 2008; 2008/0293132 (Goldman et al), published Nov. 27, 2008 and 2008/0299643 (Howard et al), published Dec. 4, 2008. In general, the microalgae are grown by introducing nutrients into a culture of microalgae, and exposing the resulting biomass to light in a cultivation zone. Problems with existing microalgae growing systems include (i) ensuring efficient mixing of the nutrients with the microalgae culture, and (ii) exposure of all areas of the biomass to light.

### BRIEF SUMMARY OF THE INVENTION

The present invention provides a bioreactor system which ensures continuous, thorough mixing of microalgae, growth medium and nutrients, and constant changing of the areas or zones of the biomass and movement of the biomass to the available light. The invention also provides a flow director for use in the bioreactor which continuously introduces nutrients into the biomass, and creates a laminar flow in the biomass ensuring that the entire biomass continually mixes and circulates in the bioreactor. Thus, all areas of the biomass are periodically moved to and regularly exposed to natural light and/or artificial light sources mounted on the bioreactor. The bioreactor system can also be used to grow other organisms benefiting from the continuous mixing of a culture of the organisms, nutrients, growth medium or exposure to light.

In its simplest form, the bioreactor system of the present invention includes a variable size bioreactor for growing an organism such as microalgae comprising a culture container having side walls, end walls and at least a partially rounded bottom wall for carrying a culture medium for the organism; at least one pipe for feeding nutrients into said culture medium in the container; and a flow director on the container bottom wall for receiving a fluid under pressure, said flow director including an elongated body having a pair of upwardly converging side walls for directing culture medium upwardly along each side wall, a bottom wall extending between bottom ends of the side walls; a first tube at the top ends of the side walls; said first tube having openings for discharging a fluid under pressure upwardly into the culture medium to establish laminar flow of the culture medium upwardly, then outwardly towards the container side walls, which direct the fluid downwardly along the container side walls, along the container bottom wall, and upwardly along the flow director side walls into the path of the upwardly flowing fluid.

### BRIEF DESCRIPTION OF THE DRAWINGS

The invention is described below in greater detail with reference to the accompanying drawings, which illustrate a preferred embodiment of the invention, and wherein:

FIG. 1 is a schematic top view of a bioreactor system in accordance with the invention;

FIG. 2 is a schematic, partly sectioned end view of the system of FIG. 1;

FIG. 3 is an isometric view of a section of a flow director used in the system of FIGS. 1 and 2;

FIG. 4 is an end view of the flow director of FIG. 4; and

FIG. 5 is a cross-sectional view of a portion of a bioreactor tank used in the system of FIGS. 1 and 2.

### DETAILED DESCRIPTION OF THE INVENTION

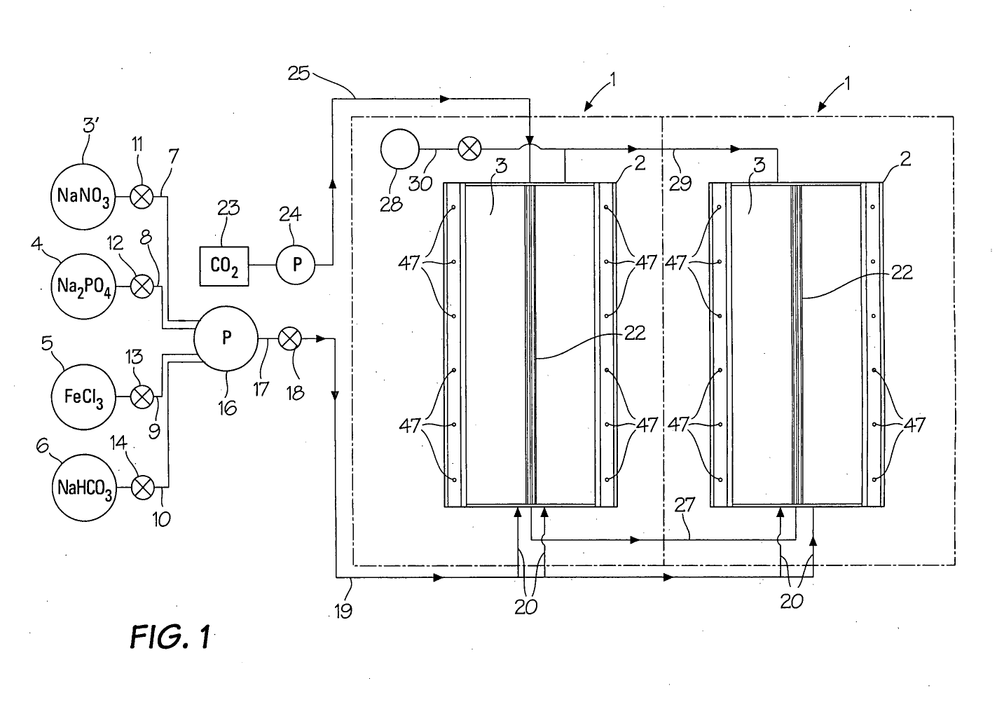

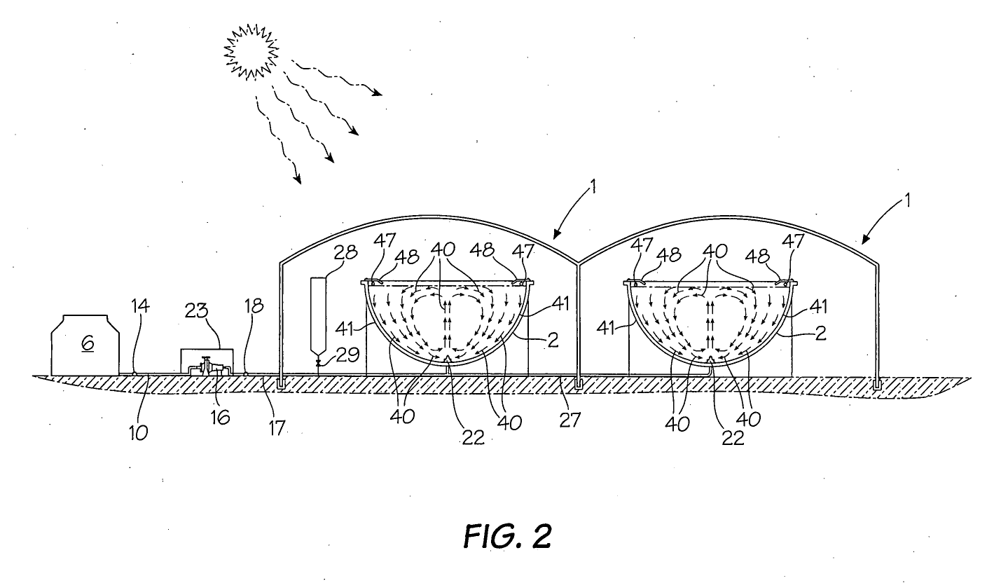

With reference to FIGS. 1 and 2, a bioreactor system in accordance with the invention includes a pair of greenhouse-like housings indicated generally at 1. It will be appreciated that such housings 1 may not be utilized in all applications or a single structure or housing could be used, and that a large bioreactor system would include many such housings 1. Each of the housings 1 contains a container defined by a pair of rectangular culture tanks 2 having parabolic cross-sectional configurations. From the following description, it will be appreciated that the cross section of the culture tank 2 can be other than parabolic. The same results can be achieved using another container such as a recess in the ground or a tank with a rectangular cross section having a horizontal bottom wall, vertical side walls and chamfered bottom corners. The tanks 2 are intended to carry a culture medium 3 consisting of water and various nutrients.

The nutrients for microalgae cultivation are variable, and may include aqueous solutions of sodium nitrate (NaNO₃) in a container 3, sodium phosphate (Na₂PO₄) in a container 4, ferric chloride (FeCl₃) in a container 5, and sodium bicarbonate (NaHCO₃) in container 6. The nutrients are fed from the tanks 3-6 via pipes 7-10 containing valves 11-14, respectively to a pump 16. From the pump 16, the nutrients flow through a pipe 17 containing a check valve 18, and pipes 19 and 20 to flow directors 22 in the tanks 2.

At the same time carbon dioxide (CO₂) is mixed with water using a mixing apparatus 23 of the type described in U.S. Pat. No. 6,209,855, issued to Craig L. Glassford on Apr. 3, 2001, which is incorporated herein by reference or an alternative apparatus for mixing the carbon dioxide with the water could be utilized. The carbon dioxide and water mixture is fed via a pump 24 and a pipe 25 to one end of the first flow director 22. The CO₂ and water mixture then proceeds via pipe 27 to the second flow director 22.

A culture of microalgae is continuously fed from a transparent culture cylinder 28 via pipe 29 into the tanks 2. The microalgae target species is used to continuously innoculate feed water introduced into headworks of the first culture tank 2. The innoculant solution is gravity fed and flow is controlled by an adjustable valve 30.

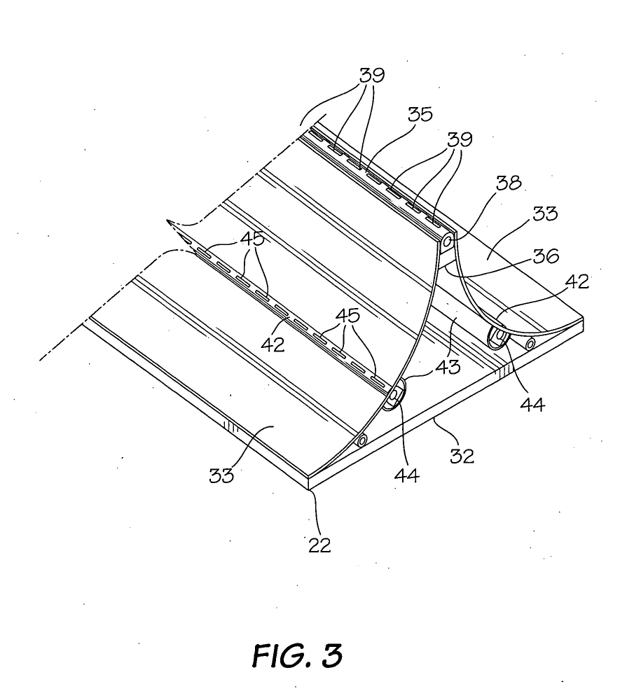

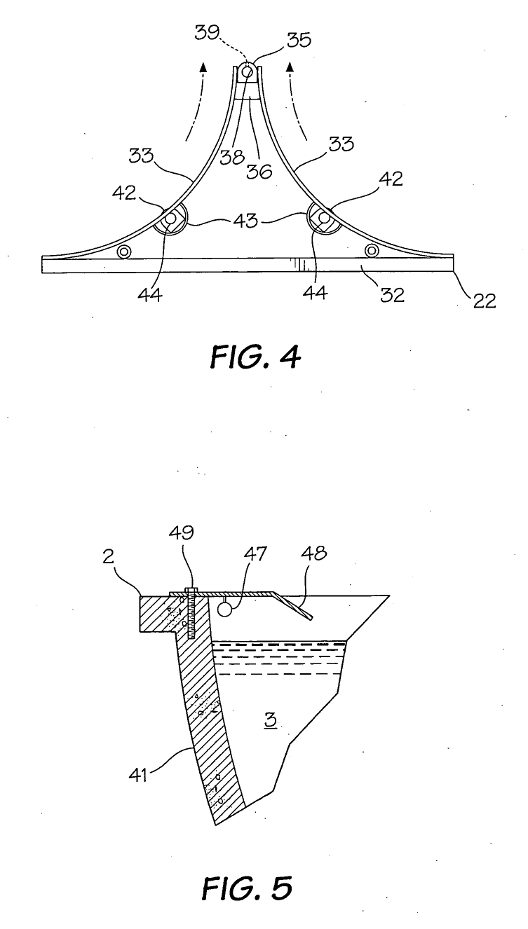

With reference to FIGS. 3 and 4, each flow director 22 includes a generally triangular body defined by a flat bottom wall 32 and a pair of side walls 33. It will be appreciated that the bottom wall of the triangular body can be defined by the bottom wall of the tank, i.e. the bottom ends of the flow director side walls can be connected directly to the tank bottom wall 22. The side walls 33 have concave outer surfaces. A flexible tube 35 extending the length of the flow director 22 is sandwiched between the top ends 36 of the side walls 33. The tube 35 is supported in the flow director by a bar 30 connected to the side walls 33 beneath the tube. The tube 35 is similar to the fine bubble aeration tube described in US Patent Publication 2008/0296789 (John N. Hinde) published Dec. 4, 2008 (incorporated herein by reference).

The tube 35 includes a central passage 38 and a plurality of slits 39 in its semicylindrical top end communicating with the passage 38. When fluid under pressure is pumped through the tube, the slits 39 act as one-way valves for discharging the fluid into the culture medium 3 in the tanks 2. The tube 35 is used to introduce the CO₂—containing water or another CO₂—containing fluid into the culture medium 3 creating laminar flow of the culture medium. As indicated by the arrows 40 in FIG. 2, the fluid discharged from the tube 35 flows upwardly from the flow director 22, then outwardly towards the sides 41 (FIGS. 2 and 5) of the tank 2, downwardly following the curved sides of the tank to the bottom ends of the sides of the flow director 22, and finally flows upwardly to join the flow from the tube 35.

At the same time, nutrients from the reservoirs 3-6 flow into tubes 42 mounted beneath the flow director side walls 33 with their outer, top ends extending through the side walls. The tubes 42 are supported in the flow director 22 by semicylindrical brackets 43. The tubes 42 are similar in structure to the tubes 35, including central passages 44 and slits 45 in their outer, top ends. The tubes 42 are used to provide a supply of nutrients under low pressure to the biomass in the tanks 2.

Thus, it will be appreciated that the mixture of microalgae, carbon dioxide and nutrients continuously circulates in laminar flow ensuring maximum mixing of the nutrients and exposure of the culture to available light from natural and/or artificial sources of light 47 mounted on the top of each side of each tank 2. As best shown in FIG. 5, the artificial lights 47 are suspended beneath stainless steel reflectors 48, which are connected to the sides 41 of the tanks 2 by bolts 49.

In summary, the apparatus described hereinbefore integrates five fundamental processes to enhance the growth of micro-algal biomass beyond normal levels throughout the entire three-dimensional capacity of the apparatus. The processes involved are (i) the utilization of gas/liquid diffusion to create a continuous, non-turbulent laminar flow movement of the entire culture medium in the tanks 2, (ii) the creation of distinct aquatic vortex growth zones, (iii) the controlled dispersion and suspension of the entire microalgal biomass, (iv) the controlled nutrient delivery to the entire microalgal biomass and (v) the controlled light exposure of the entire microalgal biomass.

By placing the flow director 22 in a precise location, at the bottom center of a cultivation tank 2 or other container with a suitable cross-sectional configuration, e.g. parabolic or rectangular with chamfered bottom corners, a vector laminar water flow creates a dynamic vortex and a controlled means of moving microalgae to the natural (sunlight) and/or artificial light sources 47. This regulates the exposure time of the microalgae population to the light source, uniformly suspends the microalgae in the culture system for maximum access to nutrients, uniformly distributes nutrients to the microalgae and manages the entire growth cycle by manipulation of the key factors required for enhanced growth of the microalgal biomass.

The use of laminar flow results in gentle movement of the microalgae to eliminate potential cell damage or stress caused by random turbulent flow. The vector laminar flow ensures maximum light penetration into the aquatic culture medium by dispersion and uniform distribution of individual algae cell bodies in the aquatic culture medium for greater exposure to the available light sources. Moreover, uniform dispersion and distribution of the nutrients in the aquatic culture improves essential nutrient availability for sustainable and optimum growth of algal biomass.

1. A bioreactor for growing an organism comprising:
a culture container having an open top end, side walls, end walls and an at least partially rounded bottom wall for carrying a culture medium for the organism;
at least one first pipe for feeding nutrients into said culture medium in the culture container;
a flow director on the bottom wall of the container for receiving a fluid under pressure, said flow director including an elongated body having a pair of upwardly converging side walls for directing culture medium upwardly long each side wall, and a bottom wall extending between bottom ends of the side walls, a first tube at top ends of the side walls, said first tube having openings for discharging a fluid under pressure upwardly into the culture medium to establish laminar flow of the culture medium upwardly, then outwardly towards the container side walls, which direct the culture medium downwardly along the container side walls, along the tank bottom wall and upwardly along the flow director side walls into the path of upwardly flowing fluid.

2. The bioreactor of claim 1, wherein said flow director side walls are concave along their lengths.

3. The bioreactor of claim 2, wherein the container side and bottom walls define a parabola.

4. The bioreactor of claim 3, wherein the flow director is located in the bottom center of the container.

5. The bioreactor of claim 4, including a first pipe for introducing said fluid under pressure into one end of said flow director first tube.

6. The bioreactor of claim 4, wherein when the organism is microalgae, the culture medium is water containing the nutrients and the fluid under pressure is a carbon dioxide in water growth medium.

7. The bioreactor of claim 6, including a pair of second pipes for introducing nutrients into the bottom of the container on each side of the flow director.

8. The bioreactor of claim 7, including second tubes in said flow director side walls for receiving nutrients from said second pipes, said second tubes extending the length of said flow director side walls, and including openings for discharging nutrients into said culture medium.

9. The bioreactor of claim 8 including artificial light sources on the upper ends of said container side walls for promoting the growth of microalgae.

10. The bioreactor of claim 9, wherein said artificial light sources include stainless reflectors extending inwardly from the upper ends of the container side walls, and artificial lights suspended from said reflectors above the culture medium.

11. A bioreactor systems for growing microalgae, comprising:
an at least partially transparent housing for admitting sunlight;
a culture tank having an open top end, side walls, end walls and at least a partially rounded bottom wall for carrying a culture medium for growing microalgae;
at least one pipe for feeding nutrients into said culture medium in the tank;
a flow director on the bottom of the tank for receiving a fluid under pressure, said flow director including an elongated body having a pair of upwardly converging side walls for directing culture medium upwardly along each side wall, and a bottom wall extending between bottom ends of the side walls, a first tube at top ends of the side walls, said first tube having openings for discharging a fluid under pressure upwardly into the culture medium to establish laminar flow of the culture medium upwardly, then outwardly towards the tank side walls, which direct the culture medium downwardly along the tank side walls, along the tank bottom wall and upwardly along the flow director side walls into the path of the upwardly flowing fluid.

12. The bioreactor of claim 10, wherein said flow director side walls are concave along their lengths.

13. The bioreactor of claim 12, wherein the tank side and bottom walls define a parabola.

14. The bioreactor of claim 12, wherein the flow director is located in the bottom center of the tank.

15. The bioreactor of claim 14, wherein when the culture medium is water containing nutrients and the fluid under pressure is a carbon dioxide in water growth medium.

16. The bioreactor of claim 15, including a first pipe for introducing the carbon dioxide and water growth medium under pressure into one end of said flow director first tube.

17. The bioreactor of claim 16, including a pair of second pipes for introducing nutrients into the bottom of the tank on each side of the flow director.

18. The bioreactor of claim 17, including second tubes in said flow director side walls for receiving nutrients from said second pipes, said second tubes extending the length of said flow director side walls, and includes openings for discharging nutrients into said culture medium.

19. The bioreactor of claim 18, including artificial light sources mounted on the upper ends of said tank side walls for promoting the growth of microalgae.

20. The bioreactor of claim 19, wherein said artificial light sources includes reflectors extending inwardly from the upper ends of the tank side walls, and artificial lights suspended from said reflectors above the culture medium.
